(19) United States
(12) Patent Application Publication
(10) Pub. No.: US 2026/0263421 A1
(43) Pub. Date: Sep. 10, 2026

(54) Method of Mitigating Radiation Injury with Geranylgeranyl Transferase Inhibitors

(71) Applicant: BioVentures, LLC, Little Rock, AR (US)

(72) Inventors: Rupak Pathak, Little Rock, AR (US); Marjan Boerma, Little Rock, AR (US)

(21) Appl. No.: 18/876,520

(22) PCT Filed: Jun. 22, 2023
(86) PCT No.: PCT/US23/25943

§ 371 Date: Dec. 18, 2024

Related U.S. Application Data
(60) Provisional application No. 63/354,299, filed on Jun. 22, 2022.

(51) Int. Cl. A61K 31/417; A61K 31/223; A61P 1/00

(52) U.S. Cl. CPC A61K 31/417; A61K 31/223; A61P 1/00

(57) ABSTRACT

A method of treating a patient with intestinal radiation injury is disclosed. The method includes administering an effective amount of a geranylgeranyl transferase inhibitor to a patient with intestinal radiation injury. In one aspect, the geranylgeranyl transferase inhibitor is GGTi-298. In another aspect, the geranylgeranyl transferase inhibitor is GGTi-2133.

### CROSS-REFERENCE TO RELATED APPLICATIONS

This application claims the benefit of U.S. Provisional Application No. 63/354,299, entitled “Mitigation of Radiation Injury By Geranylgeranyl Transferase Inhibitors” and filed on Jun. 22, 2022. The complete disclosure of said provisional application is hereby incorporated by reference.

### STATEMENT REGARDING FEDERALLY SPONSORED RESEARCH OR DEVELOPMENT

Not applicable

### BACKGROUND OF THE INVENTION

In the event of a radiation emergency, people may be exposed to ionizing radiation, and therapeutic interventions that reduce radiation injuries when administered 24+ hours after radiation exposure will be needed (i.e., radiation mitigators). While the U.S. Food and Drug Administration (FDA) has approved some therapeutic strategies that mitigate hematopoietic radiation damage, mitigators of radiation injury in other organ systems including the intestine have not yet been approved.

It would therefore be desirable to develop a mitigator of radiation injury in other organ systems. Geranylgeranyl transferase inhibitors (GGTi) are currently in clinical trials to treat patients with advanced malignancies. However, their efficacy as a mitigator of intestinal radiation damage has not yet been explored. In mouse models, the inventors have found that intraperitoneal administration of GGTi starting 24 hours after partial body irradiation (PBI, 12 Gy γ-rays with both hind legs shielded from radiation) followed by additional administration of GGTi every 48 hours thereafter mitigates intestinal damage measured on days 3.5 and 14. In addition, intraperitoneal administration of GGTi starting 24 hour after 12 Gy PBI followed by a second dose of GGTi at 72 hours altered the composition of the intestinal microbiome in radiation exposed mice. Finally, GGTi suppresses the proliferation rate of human pancreatic and prostate cancer cell lines in a time-dependent and dose-dependent manner.

### BRIEF SUMMARY OF THE INVENTION

The present invention is directed to a method of using geranylgeranyl transferase inhibitors to mitigate or treat intestinal radiation injury, including in radiation accident/terrorism scenarios and in radiation therapy. In one aspect, the method of the present invention includes administering an effective amount of a geranylgeranyl transferase inhibitor to a patient with intestinal radiation injury. As an example, the geranylgeranyl transferase inhibitor may be GGTi-298 or GGTi-2133.

In adult male C57BL/6J mice, the inventors observed that intraperitoneal administration of GGTi-2133 (commercially available, Sigma Aldrich) three times per week starting 24 hours after 12 Gy γ-ray PBI for a total of 2 weeks significantly attenuated body weight loss, and in the small intestine prevented reduction in villus height, mucosal surface area, goblet cell number, immune cell number (neutrophils and T-lymphocytes) and proliferating cell number in the crypts compared to intraperitoneal administration of vehicle (90% sterile saline, 5% DMSO, and 5% Kolliphor® EL). In addition, GGTi-298 (commercially available, MedChemExpress, Monmouth Junction, NJ) mitigated adverse effects of 12 Gy γ-ray PBI on villus height, crypt depth and crypt width. Moreover, intraperitoneal administration of GGTi-2133 24 hours after 12 Gy PBI followed by a second dose of GGTi-2133 at 72 hours significantly enhanced the number of surviving crypts in the small intestine on day 3.5, when compared to vehicle treated irradiated mice. Additionally, microbiome analysis in the cecal content on day 3.5 revealed that GGTi-2133 increased the percentage of Akkermansia muciniphila in irradiated mice. This bacterium is inversely associated with obesity, diabetes, inflammation and metabolic disorders. Lastly, PBI-induced increases in the percentage of Turicibacter and Clostridium species were mitigated by GGTi-2133. Several Clostridium species are known to be pathogenic, while an increase in Turicibacter depletes CD8+ lymphocytes in the gut, which can adversely affect gut immune function. These data indicate that GGTi is a strong mitigator of intestinal radiation injury.

Human pancreatic cancer cell lines (PANC-1, BxPC-3, PSN-1) and a human prostate cancer cell line (LNCaP) were cultured in vitro at 37° C. and 5% CO₂ and incubated with GGTi-2133 at 3 different concentrations. Cell proliferation and metabolic rate were measured with an MTT assay once a day for a total of 4 days. GGTi-2133 reduced the cell proliferation rate in all 4 cell lines in a dose-dependent manner. Moreover, in a clonogenic assay, GGTi-2133 dose-dependently reduced the number of surviving colonies of the PANC-1 and PSN-1 cell lines. Finally, GGTi-2133 reduced the metabolic activity in LNCaP cells exposed to ionizing radiation. These data indicate that GGTi reduces the proliferation of tumor cells and enhances the effects of ionizing radiation on tumor cells.

These and other features, objects and advantages of the present invention will become better understood from a consideration of the following detailed description of the preferred embodiments and appended claim in conjunction with the drawings as described following:

### BRIEF DESCRIPTION OF THE DRAWINGS

FIGS. 1A-1E are bar graphs showing that GGTi-2133 treatment significantly attenuates reduction in villus height (FIG. 1A), increase in crypt depth (FIG. 1B), decrease in mucosal surface area (MSA) (FIG. 1C), increase in enterocyte length (FIG. 1D), and increase in the length of the enterocyte nucleus (FIG. 1E) following 12 Gy PBI. FIG. 1F is a line graph showing that GGTi-2133 treatment significantly reduces body weight loss following 12 Gy PBI. Data are represented as mean+/−standard deviation (SD; n=5 to 8 mice per group). “PBI” refers to partial body irradiation, and “Sham” refers to no PBI. The notations “a & b” refer to statistically significant difference from sham-irradiation and PBI, respectively. The notation “GGTi-1” refers to GGTi at a dose of 1 mg/kg. The notation “GGTi-2.5” refers to GGTi at a dose of 2.5 mg/kg. Both sham-irradiated mice and the PBI group received intraperitoneal administration of vehicle.

FIGS. 2A-2D are photomicrographs showing immunohistochemical staining of myeloperoxidase (MPO) positive cells (neutrophils) in the intestine of sham-irradiated vehicle treated mice (FIGS. 2A), 12 Gy PBI plus vehicle treated mice (FIGS. 2B), 12 Gy PBI plus 1 mg/kg body weight GGTi treated mice (FIG. 2C), and 12 Gy PBI plus 2.5 mg/kg body weight GGTi treated mice (FIG. 2D). FIGS. 2E-2H are bar graphs that show that GGTi-2133 treatment significantly recovers neutrophils (myeloperoxidase: MPO) (FIG. 2E) and lymphocytes (CD3ε) (FIG. 2F), and decreases macrophage (CD68) infiltration in the stromal (FIG. 2G) and intraepithelial region (FIG. 2H) in the small intestine after PBI. FIG. 2E-2F show the average number of MPO (FIG. 2E) and CD3ε positive cells (FIG. 2F) in 6 randomly selected areas (each area is 0.0625 μm²). Data are represented as mean+/−SD (n=5 to 8 mice). The notation “a & b” refers to statistically significant difference from sham and PBI, respectively.

FIGS. 3A-3D are photomicrographs showing immunohistochemical staining of Proliferating Cell Nuclear Antigen (PCNA) positive cells in the intestine of sham-irradiated vehicle treated mice (FIGS. 3A), 12 Gy PBI plus vehicle treated mice (FIGS. 3B), 12 Gy PBI plus 1 mg/kg body weight GGTi treate mice (FIG. 3C), and 12 Gy PBI plus 2.5 mg/kg body weight GGTi treated mice (FIG. 3D). FIGS. 3E-3F are bar graphs that show that GGTi-2133 treatment significantly recovers crypt cell proliferation (detected by PCNA- and Ki-67) in the small intestine after PBI. Data are represented as mean+/−SD (n=5 to 8 mice). The notation “a & b” refers to statistically significant difference from sham and PBI, respectively.

FIGS. 4A-4D are photomicrographs showing Alcian blue staining of mucus secreting cells (MSCs) in the intestine of sham-irradiated vehicle treated mice (FIGS. 4A), 12 Gy PBI plus vehicle treated mice (FIGS. 4B), 12 Gy PBI plus 1 mg/kg body weight GGTi treated mice (FIG. 4C), and 12 Gy PBI plus 2.5 mg/kg body weight GGTi treated mice (FIG. 4D). FIGS. 4E-4F are bar graphs showing that GGTi-2133 treatment significantly recovers mucus secreting cells (MSCs) as detected by Alcian blue in the small intestine after PBI. Data are represented as mean+/−SD (n=5 to 8). The notation “a & b” refer to statistically significant difference from sham and PBI, respectively.

FIGS. 5A-5D are photomicrographs showing olfactomedin 4 (OLFM4) staining in the crypts of sham irradiated vehicle treated mice (FIGS. 5A), 12 Gy PBI plus vehicle treated mice (FIGS. 5B), 12 Gy PBI plus 1 mg/kg body weight GGTi treate mice (FIG. 5C), and 12 Gy PBI+2.5 mg/kg body weight GGTi treated mice (FIG. 5D). Colors represent staining intensities: yellow (dark), red (medium), and green (weak). FIG. 5E is a bar graph showing the average staining intensity for dark, medium, and weak immunoreactivity obtained by analyzing 10 randomly selected crypts under 100× magnification from each mouse. Data are presented as mean+/−SD (n=5 to 8). The notation “a & b” refer to statistically significant difference from sham and PBI, respectively.

FIG. 6A is an H&E stained small intestinal section (20× magnification) to indicate how the villus height, crypt depth, and crypt width were measured with computerized analysis. FIGS. 6B-6D are bar graphs that show that GGTi-298 treatment significantly attenuates reduction in villus height (FIG. 6B), increase in crypt depth (FIG. 6C), and increase in crypt width (FIG. 6D) following 12 Gy PBI. Data are represented as mean+/−SD. The notation “a & b” refer to statistically significant difference from sham and PBI, respectively. Both sham-irradiated mice and the PBI group received intraperitoneal administration of vehicle.

FIGS. 7A-7C are photomicrographs of crypts on day 4: without the presence of human microvascular endothelial cells (ECs) (FIG. 7A), in the presence of KLF2 wildtype ECs (FIG. 7B), and in the presence of KLF2 knockdown ECs (FIG. 7C). FIG. 7D is a bar graph that shows the average number of crypt organoids from 4 male C57BL/6J mice. Data are represented as mean+/−SD. The notation “a & b” refer to statistically significant difference from crypts without ECs and crypts with KLF2 wildtype ECs, respectively. The notation “WT” refers to wildtype, and the notation “KD” refers to knockdown.

FIGS. 8A-8C are photomicrographs of small intestine sections stained for Proliferating Cell Nuclear Antigen (PCNA) in sham-irradiated vehicle treated mice (FIGS. 8A), 12 Gy PBI plus vehicle treated mice (FIG. 8B), and mice treated with 12 Gy PBI and 2.5 mg/kg GGTi-2133 on (FIG. 8C). Cross-sections from three different mice are shown in each of FIG. 8A, FIG. 8B, and FIG. 8C. Each mouse received two intraperitoneal injections of either vehicle or 2.5 mg/kg GGTi: the first injection was administered 24 hours after PBI and the second one was administered 72 hours after irradiation. Intestinal tissue was harvested 84 hours after irradiation. FIG. 8D is a bar graph showing the average number of surviving crypts from two intestinal cross sections that were used for data analysis. Crypts were scored under 20× magnification. Data are represented as mean+/−SD (n=8 animals per group). The notation “a & b” refer to statistically significant difference from sham and PBI, respectively.

FIGS. 9A-9C are pie charts showing the distribution of different bacterial strains in the cecal content of sham-irradiated vehicle treated mice (FIGS. 9A), 12 Gy PBI plus vehicle treated mice (FIG. 9B), and mice treated with 2 Gy PBI and 2.5 mg/kg GGTi-2133 in two administrations (FIG. 9C).

FIGS. 10A-10D are line graphs that show that GGTi-2133 treatment suppressed proliferation rate and metabolic activity of cells as measured with an MTT assay in a human pancreatic epithelioid carcinoma cell line PANC-1 (FIG. 10A), a human pancreatic adenocarcinoma cell line BxPC-3 (FIG. 10B), a human pancreatic adenocarcinoma cell line (FIG. 10C), and an androgen-sensitive human prostate adenocarcinoma cell line LNCaP (FIG. 10D). FIG. 10E are photographs of petri dishes showing that GGTi-2133 treatment suppressed the colony forming ability of two human pancreatic cancer cell lines (PANC-1 and PSN-1). FIG. 10F is a bar graph showing cell proliferation and metabolic rate in the LNCaP cell line were reduced to a greater extent after combined treatment with 2 Gy radiation plus GGTi-2133 treatment, in comparison to singular treatment with either 2 Gy radiation or GGTi-2133. Data are represented as mean+/−SD (n=4).

### DETAILED DESCRIPTION OF THE INVENTION

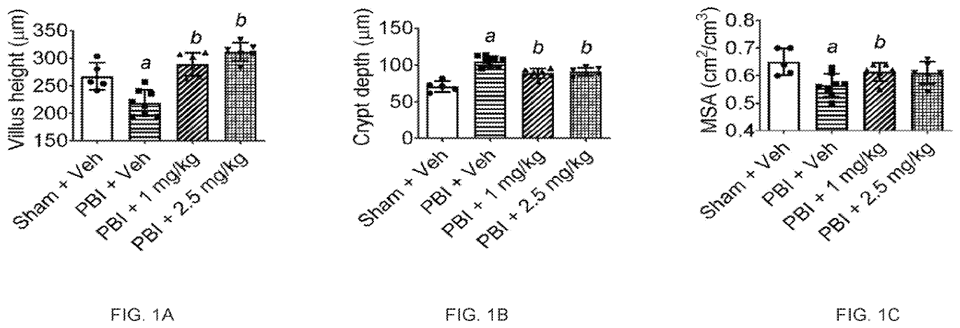

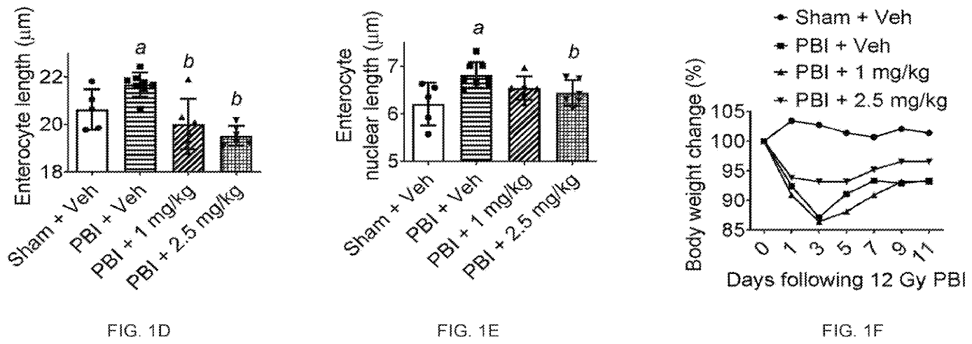

With reference to FIGS. 1A-10F, the preferred embodiments of the present invention may be described. The present invention is directed to a method of using geranylgeranyl transferase inhibitors to mitigate or treat intestinal radiation injury, including in radiation accident/terrorism scenarios and in radiation therapy. In one aspect, the method of the present invention includes administering an effective amount of a geranylgeranyl transferase inhibitor to a patient with intestinal radiation injury.

Partial body irradiation (PBI), in the event of a radiological/nuclear attack or accident, can cause damage to vital organs, including the intestine. The pathogenesis of and therapeutic strategies for mitigating gastrointestinal radiation toxicity are not well established. Damage to the endothelial cells (ECs) that form the inner lining of blood vessel contribute to the pathogenesis of intestinal toxicity. The inventors and others have shown that the loss of endothelial thrombomodulin (eTM) and impaired production of nitric oxide (NO) by endothelial nitric oxide synthase (eNOS), are mechanistically linked to the pathogenesis of intestinal radiation toxicity. Notably, both TM and eNOS are under positive transcriptional regulation of kruppel-like factor 2 (KLF2).

The mevalonate pathway regulates multiple cellular processes by synthesizing sterol isoprenoids (e.g., cholesterol) and non-sterol isoprenoids (e.g., dolichol, heme-A, isopentenyl tRNA, and ubiquinone). Geranylgeranyl transferase (GGT) is a key enzyme in the non-sterol isoprenoid pathway and is responsible for post-translational modification of various proteins. The inventors have found that pharmacological inhibitors of GGT (hereinafter, GGTi) enhance endothelial function, and prevent radiation-induced suppression of eTM, eNOS, and KLF2 in primary human ECs in culture. And most importantly, the inventors have found that co-culture of ECs with crypts promote intestinal organoid growth and that ECs required KLF2 to promote the organoid growth. The inventors hypothesized that GGTi treatment mitigates radiation injury in the intestine of mice by enhancing endothelial function after PBI. The inventors also hypothesized that mitigation of intestinal radiation injury promotes mouse survival within 30 days after total body irradiation. The inventors test their hypotheses under 2 specific aims: Aim 1—Identify the dose of GGTi that offers maximum mitigation against acute radiation toxicity; and Aim 2—Determine whether the effects of GGTi on delayed gastrointestinal is KLF2-Dependent.

The data generated from this study show that intraperitoneal administration of GGTi-2133 (Sigma) three times per week starting 24 hours after 12 Gy v-ray PBI significantly mitigates intestinal radiation toxicity and attenuates body weight loss in male C57BL/6J mice. Moreover, the inventors show that GGTi-298 also mitigates intestinal damage following 12 Gy PBI. GGTi-2133 treatment partially recovers the number of surviving crypts and alters the microbiome composition in the cecum of mice on day 3.5 after irradiation. Additionally, the inventors observed ECs promote intestinal organoid growth ex vivo in a KLF2-dependent manner. Finally, GGTi suppresses the proliferation rate and colony forming ability of human pancreatic and/or prostate cancer cell lines in culture and also enhances radiation-induced suppression of prostate cancer cell proliferation in culture. The efficacy of GGTi in mitigating various parameters of intestinal injury following PBI and reducing cancer cell line proliferation is described below.

GGTi-2133 treatment attenuates intestinal structural damage and body weight loss following PBI: In male adult C57BL/6J mice, 12 Gy PBI significantly reduced villus height, increased crypt depth, decreased mucosal surface area (MSA) and increased enterocyte length and enterocyte nuclear length as compared to the sham-irradiated group, while GGTi treatments attenuated this reduction in villus height, increase in crypt depth, decrease in MSA, and increase in enterocyte length and their nuclear length in irradiated mice, as shown in FIGS. 1A-1E. Villus height and crypt depth were measured in 30 randomly selected villi and crypts (20× magnification), MSA was measured in 5 randomly selected areas (20× magnification), and enterocyte length and the length of their nucleus were measured in 3 enterocytes/villus in 10 villi (100× magnification) for each mouse. PBI-induced body weight loss was significantly less in mice treated with GGTi (2.5 mg/kg body weight) than the mice treated with vehicle, as shown in FIG. 1F.

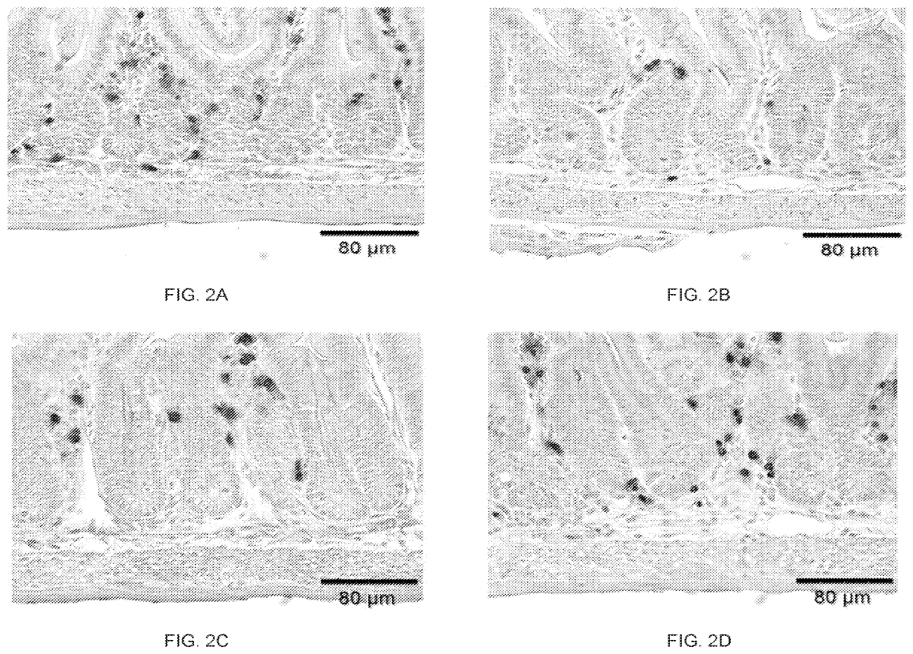

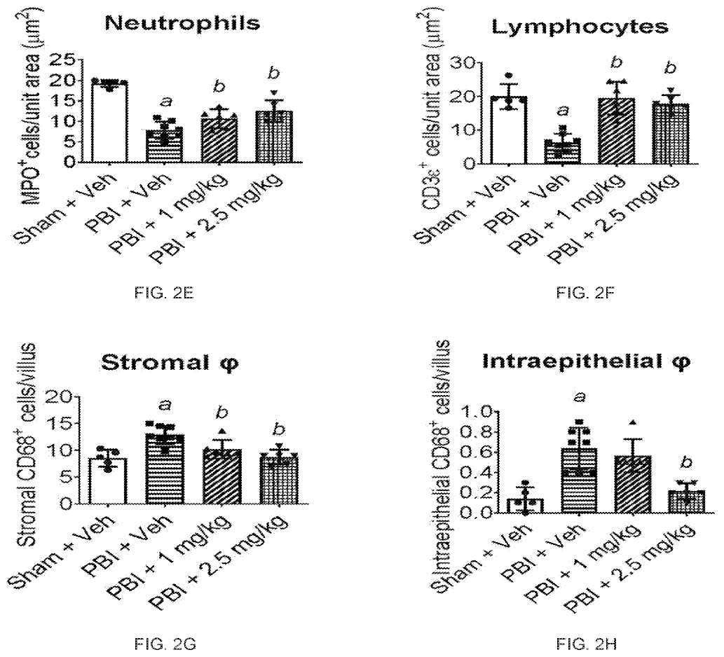

GGTi-2133 treatment accelerates recovery of the immune cells in the intestine after PBI: PBI significantly reduced the number of neutrophils and lymphocytes and increased the number of stromal and intraepithelial macrophages in the intestine as compared to sham-irradiated mice, while a significant recovery of neutrophil and lymphocyte numbers and a decrease in stromal and intraepithelial macrophage number were observed in GGTi treated irradiated mice, as shown in FIGS. 2A-2H. Neutrophils and lymphocytes were scored under 40× magnification. The average number of stromal and intraepithelial macrophage number per villus are shown in FIGS. 2G-2H. Macrophages were scored in 30 villi under 100× magnification.

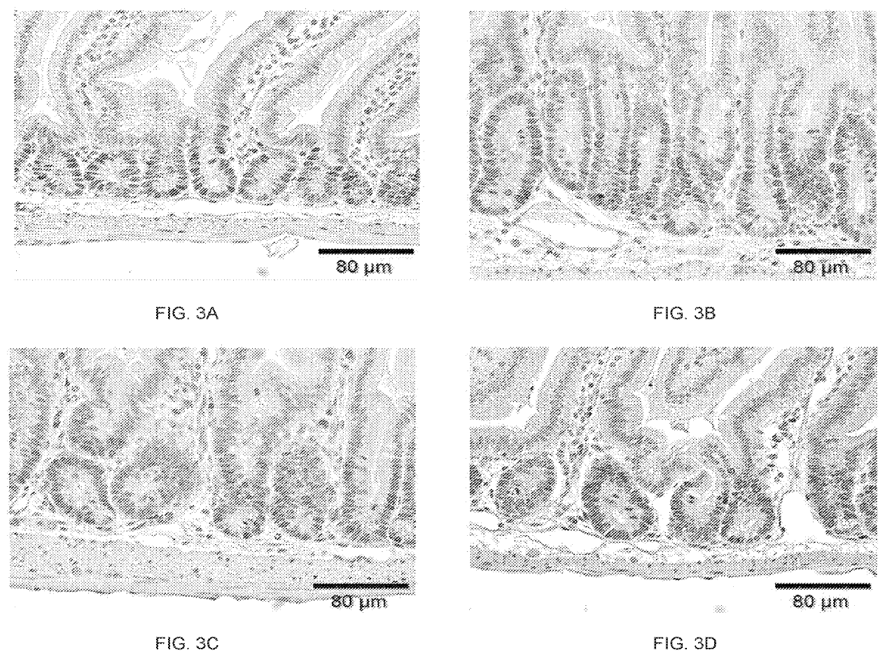

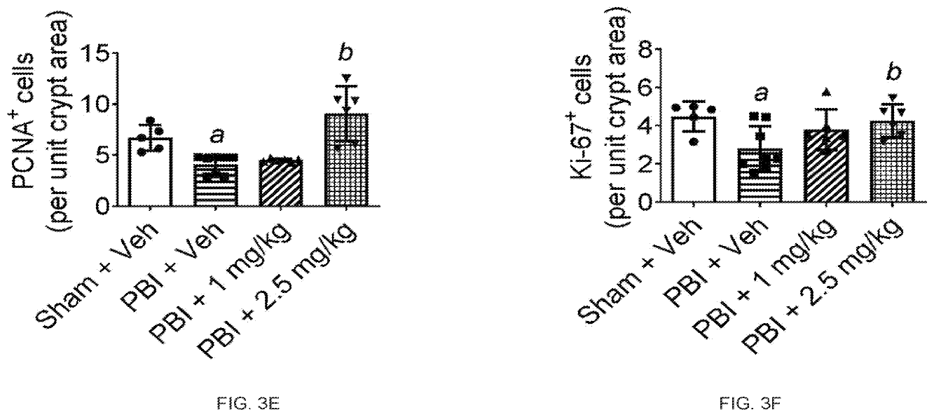

GGTi-2133 treatment increases the number of intestinal proliferating cells after PBI: Two proliferation markers (PCNA and Ki-67) were used. PBI significantly decreased the number of proliferating cells per crypt as compared to the sham-irradiated group, while treatment with 2.5 mg/kg body weight GGTi significantly increased proliferating cell number in the crypts of PBI-exposed mice, as shown in FIGS. 3A-3F. The average number of PCNA and Ki-67 positive cells in 25 randomly selected crypts are shown in FIGS. 3E-3F. Cells were scored under 40× magnification.

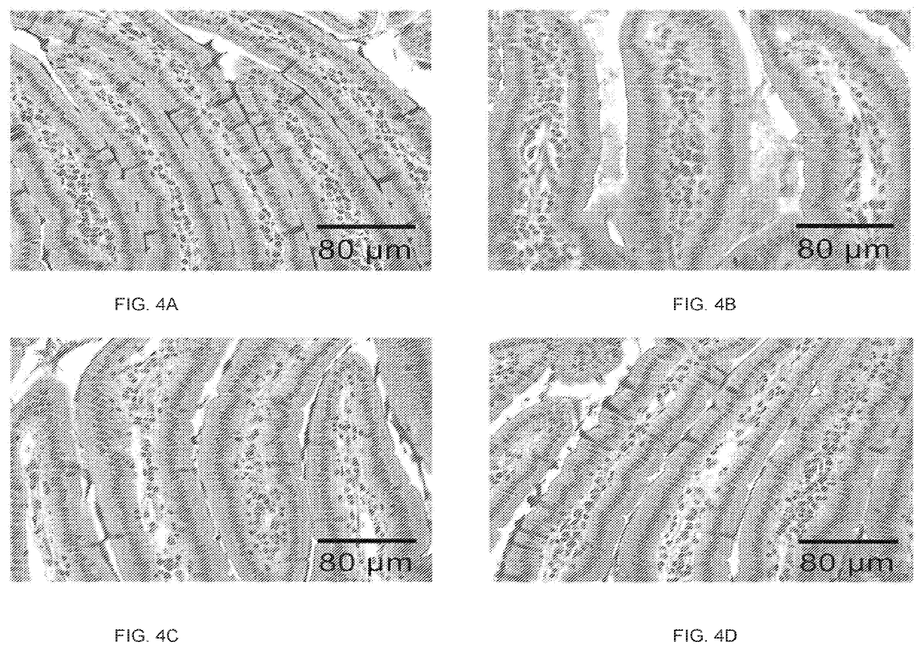

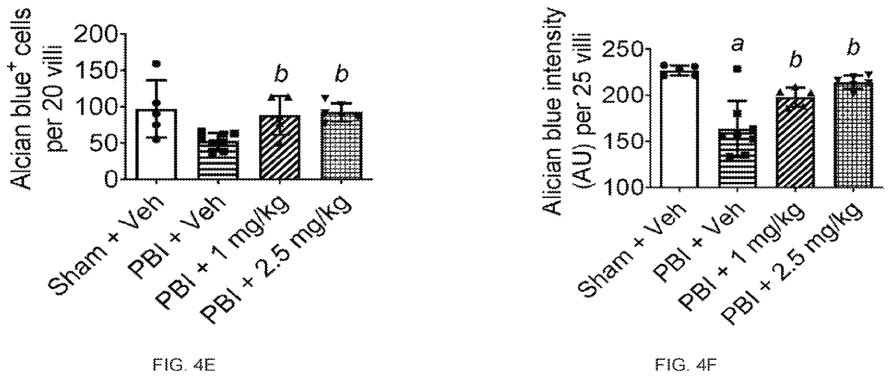

GGTi-2133 treatment enhances the number of intestinal mucus secreting cells following PBI: A significant decrease in the number of mucus secreting goblet cells was observed in the intestine following PBI as compared to the sham-irradiated group, while GGTi treatment elevated the number of goblet cells in irradiated mice, as shown in FIGS. 4A-4E. Notably, PBI also reduced the staining intensity of goblet cells, suggesting radiation suppresses mucus production, while GGTi treatment significantly enhanced the staining intensity, as shown in FIG. 4F. The average number of MSCs/20 villi and average staining intensity of Alcian blue/25 villi are shown in FIGS. 4E-4F. Cells were scored under 20× magnification.

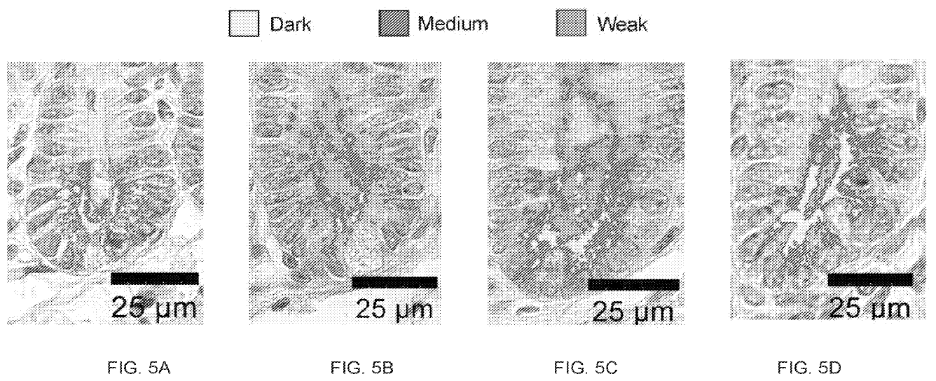

GGTi-2133 increases the expression of intestinal stem cell marker olfactomedin 4 (OLFM4) after PBI: The inventors assayed OLFM4 protein with immunohistochemistry and computerized image analysis to classify the immunoreactive regions as “dark,” “medium,” or “weak” based on staining intensity. PBI significantly decreased the area of dark OLFM4 staining, suggesting a decrease in the number of stem cells compared to sham irradiation. GGTi-2133 treatment restored OLFM4 staining in irradiated animals (FIGS. 5A-5E). FIGS. 5A-5E show that expression of the stem cell marker olfactomedin 4 (OLFM4), as examined with immunohistochemistry in the small intestine is reduced at 2 weeks after 12-Gy γ-ray PBI and increased after GGTi treatment.

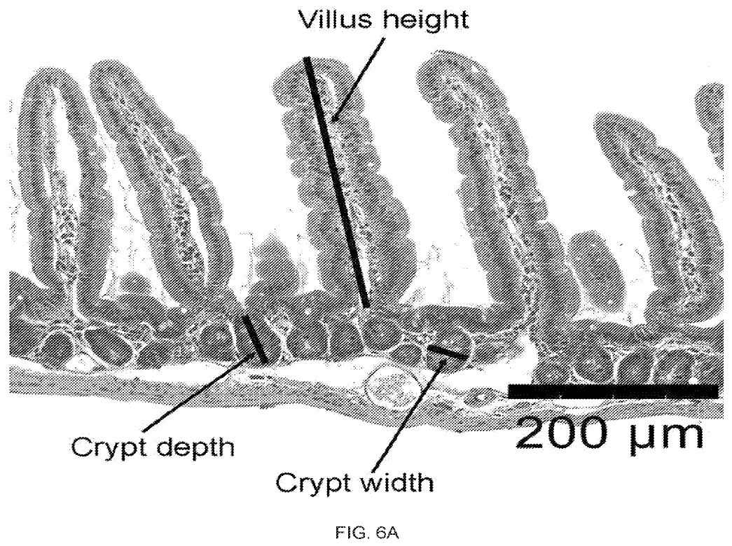

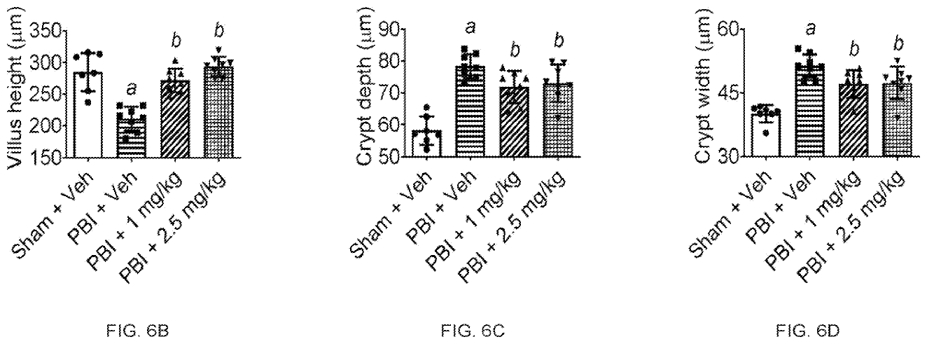

GGTi-298 treatment attenuates intestinal structural damage following PBI: In order to ensure another formulation of GGTi provides similar mitigation against intestinal damage following PBI, the inventors used GGTi-298 (MedChemExpress). In male adult C57BL/6J mice, 12 Gy PBI significantly reduced villus height, and increased crypt depth and width as compared to the sham-irradiated group, while GGTi treatments (5 or 10 mg/kg body weight) attenuated this reduction in villus height, and increase in crypt depth and width in irradiated mice, as shown in FIGS. 6A-6D. Villus height, and crypt depth and width were measured in 30 randomly selected villi and crypts (20× magnification) for each mouse. Thus, FIGS. 6B-6D show that GGTi-298, another GGTi formulation is also effective in mitigating intestinal radiation injury in the inventors' mouse model.

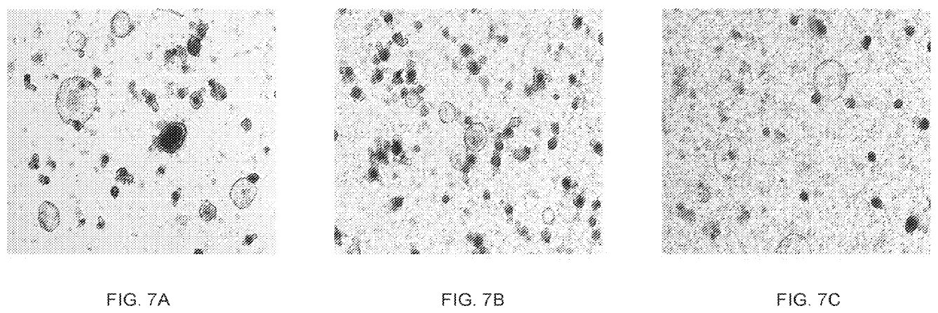

ECs lacking the KLF2 gene do not promote intestinal organoid growth: The inventors hypothesized that GGTi mitigates radiation injury at least in part by improving the function of endothelial cells (ECs). FIGS. 7A-7D show that ECs promote organoid growth in a KLF2-dependent manner. Mouse small intestinal crypts (250/well in 24-well plate) were used to grow in vitro organoids for 4 days with Kruppel-like Factor 2 (KLF2) wildtype or KLF2 knockdown human microvascular ECs (NFKB-TIME; ATCC; CRL-4049, 100,000 cells/well). Intestinal crypts organoid growth was significantly induced by the presence of ECs, and the ECs required KLF2 to promote the organoid growth. Mouse intestinal organoids grown in the presence of human microvascular ECs (NFKB-TIME; ATCC; CRL-4049) significantly increases (p=0.0001) the number of organoids. Incubation of crypts with ECs lacking the KLF2 gene does not promote intestinal organoid number when compared to the number of organoids grown without ECs, as shown in FIGS. 7A-7D.

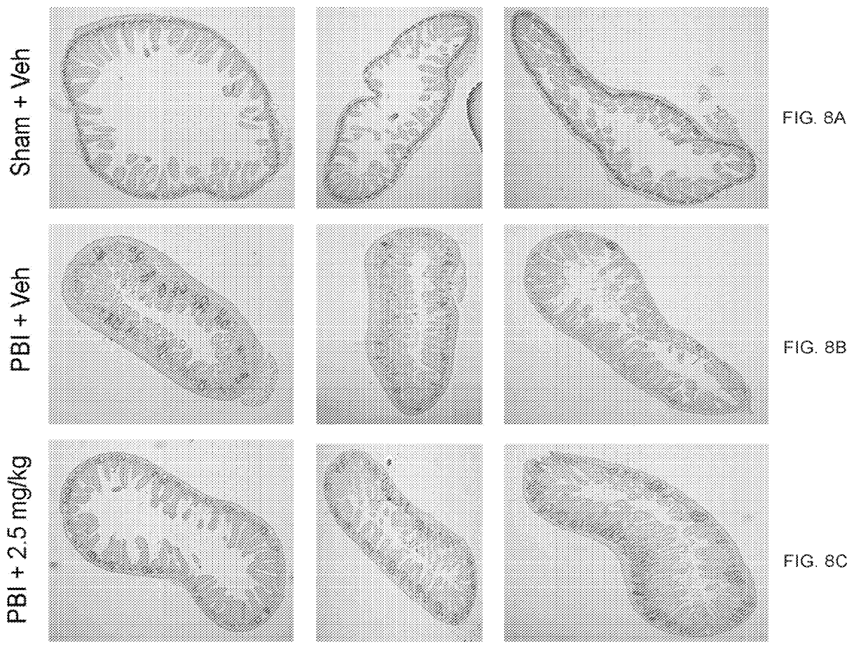

GGTi-2133 treatment enhances surviving crypt number on day 3.5 after PBI: Male C57BL/6J mice were exposed to 12 Gy PBI and administered GGTi-2133 (2.5 mg/kg) at 24 hours and 72 hours after irradiation. A specimen of the jejunum was collected on day 3.5 after irradiation. The presence of more than five PCNA positive cells grouped together in a crypt was recorded as a surviving crypt. A significant decrease (p<0.0001) in the number of surviving crypts was observed following irradiation, as compared to the sham-irradiated group, while GGTi-2133 (2.5 mg/kg) treatment significantly (p=0.002) increased the number of surviving crypts, as shown in FIGS. 8A-8D. FIGS. 8A-8D show that GGTi-2133 treatment significantly increases the number of surviving crypts in the small intestine on day 3.5 after 12 Gy PBI in a mouse model. Specimens of small intestine were stained for Proliferating Cell Nuclear Antigen (PCNA) to identify proliferating cells. As described above, intestinal crypts with at least 5 PCNA positive cells were considered as surviving crypts.

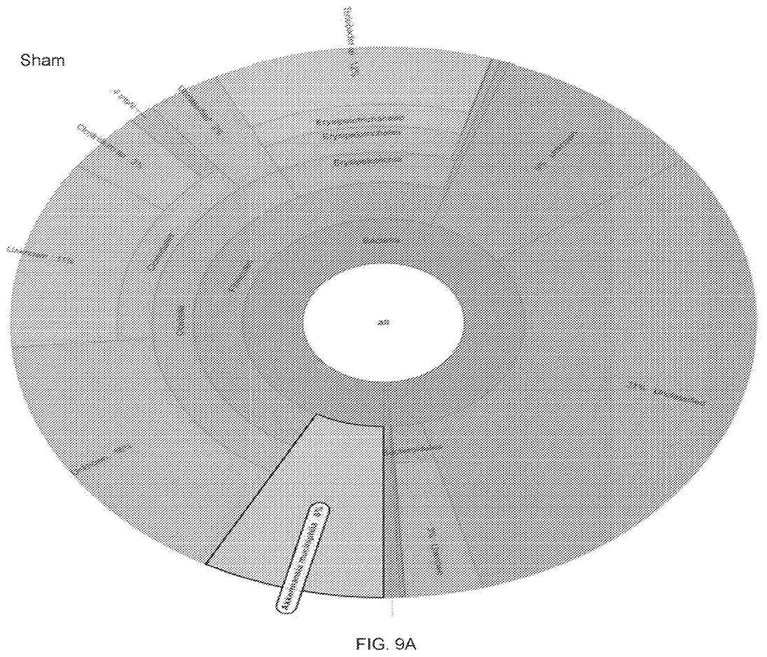

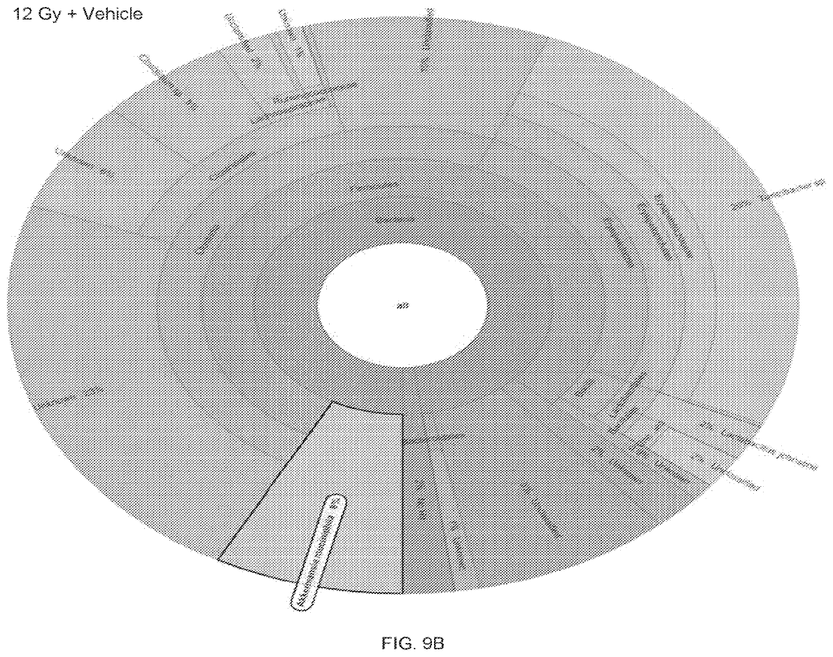

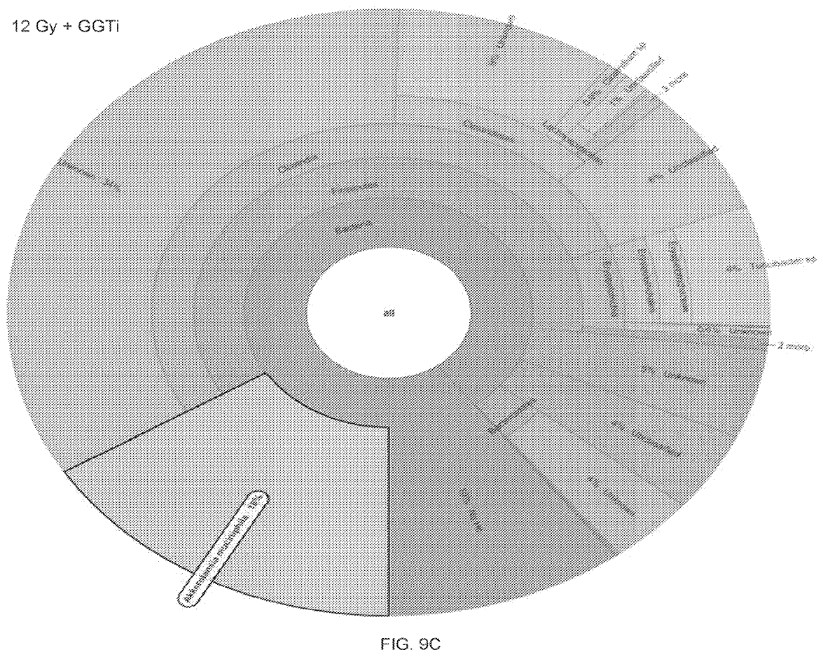

GGTi-2133 alters the percent of 3 bacterial species in the cecal content of mice on day 3.5 after PBI: Male C57BL/6J mice were exposed to 12 Gy PBI and administered GGTi-2133 (2.5 mg/kg) at 24 hours and 72 hours after irradiation. A sample of cecal content was aseptically collected 3.5 days after PBI. Microbiome analysis reveals GTTi treatment enhances Akkermansia muciniphila, while it suppresses Turicibacter sp. and Clostridium sp., in irradiated mice, as shown in FIGS. 9A-9C. There were 8 mice in each group. In particular, FIGS. 9A-9C show that GGTi-2133 (2.5 mg/kg) treatment 24 and 72 hours after 12 Gy PBI in mice alters the percentage of 3 bacterial strains in the cecal content. GGTi-2133 increased the percentage of Akkermansia muciniphila in irradiated mice. The percentage of Turicibacter and Clostridium species were increased in PBI plus vehicle treated mice as compared to sham-irradiated vehicle-treated mice. The percentage of Turicibacter and Clostridium species were reduced in PBI plus GGTi-2133 treated mice compared to PBI plus vehicle treated mice.

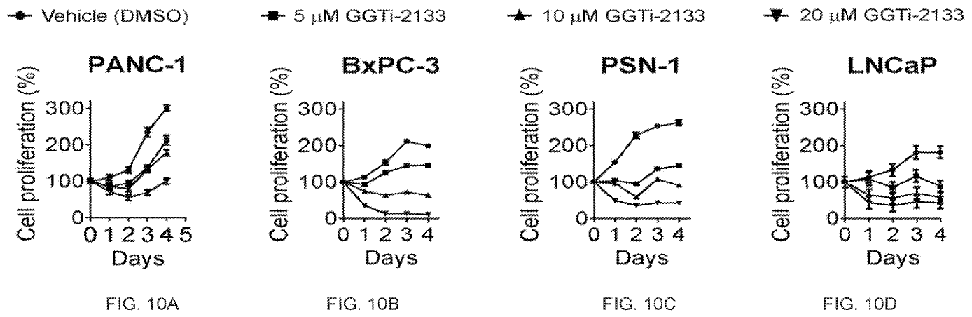

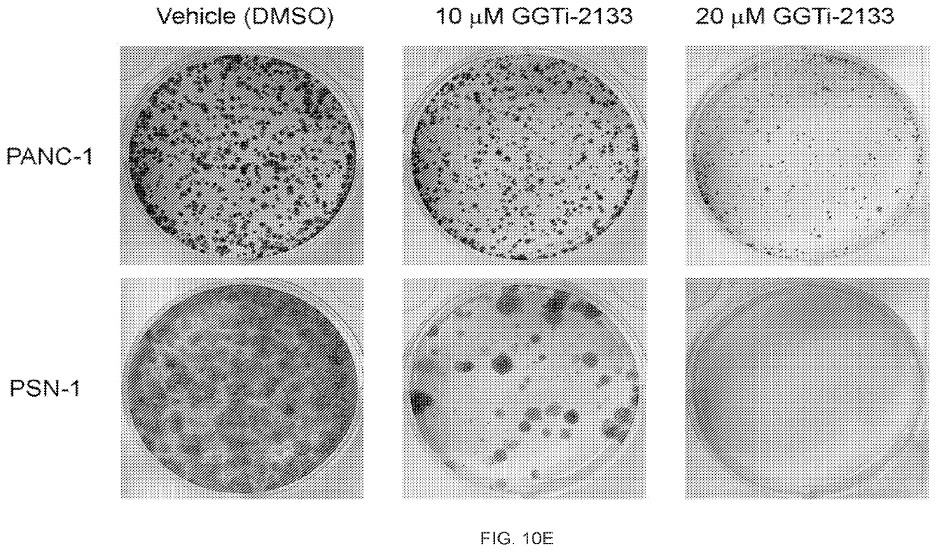

GGTi-2133 treatment suppresses the proliferation rate and colony forming ability while further enhancing the radiation-induced decline in proliferation rate of human pancreatic and/or prostate cancer cells: GGTi-2133 treatment suppresses the proliferation rate and metabolic activity of 3 pancreatic cancer cell lines (PANC-1, BxPC-3, and PSN-1) and 1 prostate cancer cell line (LNCaP) in a time-dependent and dose-dependent manner, suppresses the colony forming ability of PANC-1 and PSN-1 cell lines in a dose-dependent manner, and enhances the radiation-induced decline in proliferation rate and metabolic activity in LNCaP cells in culture, as shown in FIGS. 10A-10F. These findings suggest that GGTi has potent antitumor activities for different cancer types. Therefore, if used as a mitigator of intestinal radiation injury from radiation therapy, GGTi may not negatively impact tumor control by radiation.

Based on scientific literature, it is understood that GGTi-2418 can be administered safely to humans in doses up to 2,060 mg/m² (54 mg/kg body weight) on days 1-5 of each 21-day cycle. By using a body surface conversion factor, 54 mg/kg in humans compares to 660 mg/kg in mice. In humans, GGTi can generally be administered in an effective amount with a dose escalation from about 200 mg/m² to 2060 mg/m² per day on days 1-5 of each 21-day cycle. In rats, the maximum tolerated dose for GGTi is 200 mg/kg for a single dosing or 150 mg/kg/day for 7 consecutive days. In addition to intraperitoneal administration, oral gavage, intravenous infusion, and subcutaneous administration are other acceptable modes of administration. While GGTi-298 and GGTi-2133 are the preferred GGTis in the method of the present invention, use of other GGTis are expected to show the same effects although the magnitude of the effects may not be as great.

The methods of using GGTi as mitigator of intestinal radiation injury as described herein may be utilized in scenarios of radiation exposure due to accidents or malicious activities, or in scenarios of abdominal radiation therapy in cancer patients.

MILESTONES ACHIEVED: The inventors have completed studies to investigate intestinal acute toxicity following PBI with two different formulation of GGTi (FIGS. 1A-6D and 8A-9C). The delayed toxicity studies under specific aim 2 are ongoing. The inventors developed KLF2-knockdown stable ECs and used them to show that endothelial KLF2 is required for ECs to promote the growth of intestinal crypt organoids in vitro (FIG. 7A-D), emphasizing the importance of KLF2 in ECs for intestinal recovery. The inventors have shown that GGTi reduces the proliferation rate and enhances the cytotoxic effects of radiation in human cancer lines (FIG. 10A-F), suggesting that GGTi does not promote cancer cell growth.

PLAN FOR EXPERIMENTS: The data show that intraperitoneal administration of GGTi three times per week starting 24 h after 12 Gy γ-ray PBI significantly attenuated body weight loss, decrease in villus height, decrease in mucosal surface area, decrease in goblet cell number, decrease in immune cells (neutrophils and T-lymphocytes), decrease in proliferating cell number in the crypts, increase in crypt depth, and increase in stromal and intraepithelial macrophage number. These data indicate that GGTi is a strong mitigator of intestinal radiation injury. However, in contrast to our prior hypothesis that GGTi mainly acts via improving EC function, GGTi may also mediate direct effects on the epithelium or intestinal epithelial stem cells (ISCs). The inventors hypothesize that following irradiation, GGTi treatment promotes survival and mitigates intestinal damage as a result of direct effects of GGTi on ISCs and indirect outcomes via promoting EC function. Moreover, while not yet tested, the inventors expect that the significant mitigation of intestinal radiation injury by GGTi will provide a survival benefit following radiation. The inventors propose two aims to test these hypotheses.

Aim 1—Determine the dose of GGTi that offers maximum mitigation against radiation lethality: To further investigate GGTi as radiation mitigators, the inventors will establish that GGTi provides post-irradiation survival benefit. The inventors will optimize the GGTi dose that provides maximum survival benefit in adult male and female C57BL/6J mice exposed to total body irradiation (TBI, doses from 7 to 9 Gy γ-rays) and PBI (17 to 18.5 Gy γ-rays). The inventors have extensive experience with these radiation models. Twenty-four hours after irradiation, the inventors will administer GGTi-2133 (Sigma) or vehicle via intraperitoneal injection. The inventors will test 4 doses of GGTi-2133, administered 3 times per week up to 30 days after irradiation.

Aim 1 Research plan: Mouse TBI and PBI model: The inventors will use male and female, 8- to 12-week-old C57BL/6J mice (The Jackson Laboratory, stock no: 000664). Following 2 weeks of quarantine, radiation experiments will be started. The inventors will expose unanesthetized mice to a single dose of 7, 7.5, 8, 8.5 or 9 Gy γ-rays TBI or 17, 17.5, 18, or 18.5 Gy γ-rays PBI (with both hind legs shielded) using a cesium-137 source (Mark 1, Model 68A, JL Shepherd). In the inventors' experience, these radiation doses cause lethality in the range of 30 to 100% in C57BL/6J mice. To create 30-day survival curves, mice (n=10 mice/group) will be monitored twice/day for 30 days post-irradiation.

GGTi treatment: GGTi-2133 (Millipore Sigma) will be dissolved in a vehicle of 90% sterile saline, 5% DMSO, and 5% Kolliphor® EL (Sigma-Aldrich) and administered by intraperitoneal injection 3 times per week. GGTi-2133 treatment will begin 24 h after TBI or PBI, and continue until euthanize. Based on prior results described above, 4 doses (0, 2.5, 5, and 10 mg/kg body weight) will be tested.

Aim 2—Define if GGTi-mediated mitigation of epithelial radiation injury is a direct effect on ISCs or an endothelial-dependent consequence. Rationale and hypothesis: Radiation-induced loss of ISCs disrupts epithelial regeneration, leading to disintegration of the mucosal barrier. A number of prior studies have shown that molecules protecting directly the ISCs reduce intestinal radiation damage. The inventors and others have shown that protection to the stromal cells promotes ISC regeneration following radiation. In this regard, work by the inventors has shown that ECs in the intestinal stroma play a crucial role in suppressing radiation damage. The inventors found that ECs with wild-type KLF2, not ECs with KLF2 knockdown, promote intestinal organoid growth when in co-culture with primary crypts. The inventors will use this organoid model to test the hypothesis that GGTi mitigates radiation damage in the mucosal epithelial layer by directly enhancing ISC regeneration and by promoting endothelial function.

In prior studies, the inventors have shown that co-culturing intestinal organoids with human microvascular ECs promotes organoid growth. The inventors will use this co-culture model to determine if GGTi effects on the intestinal epithelium are endothelial dependent. Crypts in the presence and absence of mouse intestinal microvascular ECs (MIMECs; Cell Biologics) will be irradiated and grown in organoid culture media with and without GGTi. In addition, to determine if GGTi-mediated protection is endothelial KLF2-dependent, the inventors will co-culture crypts with wild-type or KLF2 knockdown MIMECs. Impact: The proposed work will provide critical mechanistic insight to develop an intestinal radiation mitigation strategy using GGTi.

Aim 2 Research plan: Crypt isolation and organoid culture: Segments of the small intestine will be collected from unirradiated adult male and female C57BL/6J mice (n=5), and intestinal crypts will be isolated by treatment with Gentle Cell Dissociation Reagent (StemCell Technologies). Suspension with equal numbers of isolated crypt will be mixed 1:1 (vol/vol) with Matrigel (BD Biosciences) and plated in 48-well plates. Intesticult™ OGM Mouse Basal Medium (StemCell Technologies) will be added and replaced every 2-3 days. The inventors will collect intestine from 5 unirradiated mice to isolate crypts, and crypts will be seeded in duplicate per mouse per treatment group.

Organoid irradiation and GGTi treatment: On day 5, organoids will be exposed to 3, 4, 5 and 6 Gy γ-rays and incubated for an additional 3 days in Intesticult™ OGM Mouse Basal Medium with various concentrations of GGTi (0, 2.5, 5, and 10 μM). Organoid number, budding and size will be determined on day 8.

Generation of KLF2-knockdown stable cell lines: KLF2 knockdown will be achieved with short hairpin RNAs (shRNAs), which will allow the inventors to generate and maintain stable KLF2-deficient MIMEC lines. MIMECs (Cell Biologics) will be stably transfected with either shRNA KLF2 plasmid (Santa Cruz) or control shRNA, using Lipofectin (Invitrogen).

Mouse irradiation, crypt isolation, and co-culture of crypts with ECs: Mice (n=5) will be exposed to 6, 7 and 8 Gy γ-ray TBI and crypts will be isolated on day 4 following TBI as described above. Crypts will be co-cultured with KLF2 wild-type or knockdown ECs for 8 days with or without GGTi (0, 2.5, 5, and 10 μM), and organoid growth will be measured as above.

Expected Results, Interpretation, Pitfalls, and Alternative Approaches: The inventors expect GGTi to mitigate TBI-and PBI-induced radiation lethality and to enhance organoid growth ex vivo. Because the inventors expect GGTi to act in a KLF2-dependent manner, the inventors predict that GGTi will not promote organoid growth when crypts will be co-cultured with KLF2 knockdown ECs.

Pitfalls and alternative approaches: The proliferation rate of MIMECs can be compromised following transfection, in that case the inventors will optimize the growth media, such as with a higher percentage of FBS. TBI-induced hematopoietic damage is a crucial determinant of survivability of mice. If the inventors fail to see significant lethality protection with GGTi, the inventors will inject FDA-approved radiation mitigator G-CSF (10 μg/kg body weight/day; 3 times/week; intraperitoneal injection), an inducer of hematopoietic stem cells, in combination with GGTi after TBI.

The present invention has been described with reference to certain preferred and alternative embodiments that are intended to be exemplary only and not limiting to the full scope of the present invention as set forth in the appended claims.

We claim:

1. A method of treating a patient with intestinal radiation injury, said method comprising administering an effective amount of a geranylgeranyl transferase inhibitor to said patient with intestinal radiation injury.

2. The method of claim 1, wherein said geranylgeranyl transferase inhibitor is GGTi-298.

3. The method of claim 1, wherein said geranylgeranyl transferase inhibitor is GGTi-2133.

4. The method of claim 1, wherein said effective amount is no more than 200 mg/kg in a single dose.

5. The method of claim 1, wherein said effective amount is no more than 150 mg/kg/day for seven consecutive days.

6. The method of claim 1, wherein said effective amount is 200 mg/m²/day to 2060 mg/m²/day.

7. The method of claim 1, wherein said administration is performed by intraperitoneal injection.

8. The method of claim 1, wherein said administration is performed by intravenous infusion.

9. The method of claim 1, wherein said administration is performed by subcutaneous injection.
